(19) United States
(12) Patent Application Publication
(10) Pub. No.: US 2007/0185490 A1
(43) Pub. Date: Aug. 9, 2007

(54) Percutaneous interspinous distraction device and method

(76) Inventor: Dante Implicito, Franklin Lakes, NJ (US)

Correspondence Address: KENYON & KENYON LLP, 1500 K STREET N.W., SUITE 700, WASHINGTON, DC, 20005, US

(21) Appl. No.: 11/655,962

(22) Filed: Jan. 22, 2007

Related U.S. Application Data
(60) Provisional application No. 60/763,352, filed on Jan. 31, 2006.

(51) Int. Cl. A61F 2/30

(52) U.S. Cl. 606/61

(57) ABSTRACT

An interspinous distraction kit is provided with a distal anchor comprising a locking section moveable from a radially un-extended position in a non-deployed configuration to a radially extended position in a deployed configuration. A method for maintaining a space between adjacent spinous processes by unilateral access to the spinous processes is also provided.

### CROSS-REFERENCE TO RELATED APPLICATIONS

This application claims priority to U.S. Provisional Application No. 60/763,352, filed on Jan. 31, 2006, which is incorporated by reference herein.

### BACKGROUND OF THE INVENTION

Many spinal disorders are considered posture-dependent conditions in which symptoms such as leg and back pain, cramping and numbness are exacerbated during extension of the spine and relieved during flexion of the spine. The posture-dependent nature of such spinal disorders is based on dimensions of the spinal canal and neural foramen increasing in flexion of the spine and decreasing in extension. The mechanism of this dynamic process is based on both the deformation of soft tissues and the relative position of osseous structures. Specifically, during extension, the ligamentum flavum buckles anteriorly into the spinal canal and lateral recess, and the posterior annulus fibrosis bulges posteriorly into the spinal canal and lateral recesses. All these mechanisms are reversed in flexion.

Because the symptoms of some posture-dependent spinal conditions improve in flexion and worsen in extension, several implantable devices have been developed for a wide variety of indications that are placed between adjacent spinal processes to prevent narrowing of the spinal canal and foramina in extension, yet allow flexion, axial rotation and lateral bending. One such device is the X-STOP interspinous process distraction device indicated for lumbar neurogenic intermittent claudication secondary to lumbar spinal stenosis. The X-STOP device has a main body, which includes a spacer and a fixed proximal wing, and a free distal wing. In use, the main body is inserted through the interspinous ligament into the interspinous space and the fixed wing is secured against the proximal surface of the spinous processes. The free distal wing is then locked into the main body and secured against the distal surface of the spinous processes. A disadvantage with this type of device is that the free wing must be attached to a wing insertion instrument and the instrument must access the contralateral side of the spinous processes in order to lock the free distal wing into the main body of the device. A midline bilateral incision must be made in order for the instrument to access the contralateral side, which requires stripping of soft tissue to expose the midline structures. As such, there is a need in the art for a less invasive and less destructive method and device for maintainin a space between adjacent spinous processes.

### SUMMARY OF THE INVENTION

The present invention provides an interspinous distraction kit comprising a spacer configured to be placed between adjacent spinous processes and a distal anchor configured to abut the distal side of the spinous processes and prevent side-to-side and anterior to posterior migration of the implanted spacer. The interspinous distraction kit can also include a proximal anchor configured to abut the proximal side of the spinous processes and also prevent side-to-side and anterior to posterior migration of the implanted spacer.

In an embodiment, the present invention provides an interspinous distraction kit comprising a distal anchor comprising a locking section moveable from a radially un-extended position in a non-deployed configuration to a radially extended position in a deployed configuration. The interspinous distraction kit further comprises a spacer having an inner surface defining an opening therethrough, the spacer configured to be positioned between adjacent spinous processes to maintain a space therebetween.

In another embodiment, the present invention provides an interspinous distraction kit comprising a distal anchor comprising a first member pivotally coupled to a second member. The first member is moveable from a radially un-extended position in a non-deployed configuration to a radially extended position in a deployed configuration. The interspinous distraction kit further comprises a spacer having an inner surface defining an opening therethrough, the spacer configured to be positioned between adjacent spinous processes to maintain a space therebetween.

In an embodiment, the present invention provides an interspinous distraction kit including a distal anchor comprising a tubular member axially splitting into at least two limbs, a wedge, and a spacer configured to be positioned between adjacent spinous processes to maintain a space therebetween.

In another embodiment, the present invention provides an interspinous distraction assembly comprising a tubular member axially splitting into at least a pair of proximal limbs and at least a pair of distal limbs, the tubular member defining a bore extending therethrough. The interspinous distraction kit further comprises a distal wedge, a proximal wedge defining an opening therethrough, and a spacer positioned on the tubular member between the proximal limbs and the distal limbs

In another embodiment, the present invention provides a method for maintaining a space between adjacent spinous processes comprising incising a lateral portion of a patient's body and advancing an interspinous distraction assembly towards the midline of the patient's body. The interspinous distraction assembly comprises a spacer, a distal anchor, and a proximal anchor. The method further comprises positioning the spacer in the interspinous space between adjacent spinous processes and deploying the distal anchor to secure the distal anchor against the distal surface of the adjacent spinous processes. The method further comprises deploying the proximal anchor after deployment of the distal anchor to secure the proximal anchor against the proximal surface of the adjacent spinous processes.

### BRIEF DESCRIPTION OF THE DRAWINGS

The present invention will become more fully understood from the detailed description given hereinbelow and the accompanying drawings which are given by way of illustration only and wherein:

FIG. 1 is a side view of an embodiment of a distal anchor in a non-deployed configuration.

FIG. 2 is a bottom view of a first member of the distal anchor of FIG. 1.

FIG. 3 is a side view of a first member of the distal anchor of FIG. 1.

FIG. 4 is a bottom view of a second member of the distal anchor of FIG. 1.

FIG. 5 is a cross-sectional view of a locking portion of a second member of the distal anchor of FIG. 1.

FIG. 6 is a top view of a distal anchor in partial cutaway showing an insertion tool inserted into the distal anchor according to an embodiment of the present invention.

FIG. 7 is a side view of the distal anchor of FIG. 6 in partial cutaway showing an insertion tool inserted into the distal anchor according to an embodiment of the present invention.

FIG. 8 is a side view of the distal anchor of FIG. 6 in partial cutaway showing an insertion tool inserted into the distal anchor according to an embodiment of the present invention.

FIG. 9 is a side view of the distal anchor of FIG. 6 in partial cutaway showing an insertion tool inserted into the distal anchor according to an embodiment of the present invention.

FIG. 10 is a side view of a distal anchor according to an embodiment of the present invention in a non-deployed configuration.

FIG. 11 is a side view of the distal anchor of FIG. 10 in a deployed configuration.

FIG. 12 is a perspective view of a spacer according to an embodiment of the present invention.

FIG. 13 is a side view of a proximal anchor in a non-deployed configuration according to an embodiment of the present invention.

FIG. 14 is a side view of the proximal anchor of FIG. 13 in a deployed configuration.

FIG. 15 is a perspective view of a proximal anchor in a deployed configuration showing an insertion tool inserted in the proximal anchor according to another embodiment of the present invention.

FIG. 16 is a bottom view of the proximal anchor of FIG. 15.

FIG. 17 is a side view of the interspinous distraction assembly according to an embodiment of the present invention in a non-deployed configuration.

FIG. 18 is a side view of the interspinous distraction assembly of FIG. 17 according to an embodiment of the present invention in a deployed configuration.

FIG. 19 is a side view of an interspinous distraction assembly according to another embodiment of the present invention in a deployed configuration.

FIG. 20 is a side view of an insertion tool according to an embodiment of the present invention.

FIG. 21 is a side view of a serial dilator according to an embodiment of the present invention.

FIG. 22 is a schematic illustration of a patient lying in the lateral decubitus position.

FIG. 23 is a side view of an interspinous assembly according to the present invention with the distal and proximal anchors in a non-deployed configuration.

FIG. 24 is a side view of the interspinous assembly of FIG. 23 illustrating the distal anchor moving towards a deployed configuration.

FIG. 25 is a side view of the interspinous assembly of FIG. 23 with the distal anchor in a deployed configuration.

FIG. 26 is a side view of the interspinous assembly of FIG. 23 with the distal and proximal anchors in a deployed configuration.

### DETAILED DESCRIPTION OF THE INVENTION

The present invention provides an interspinous distraction kit comprising a distal anchor having a locking section configured to lock against the distal side of spinous processes to secure a spacer in the interspinous space. The locking section is moveable from a radially un-extended position in a non-deployed configuration to a radially extended position in a deployed configuration. In an embodiment of a distal anchor, the locking section includes a first member that is pivotally coupled to a second member.

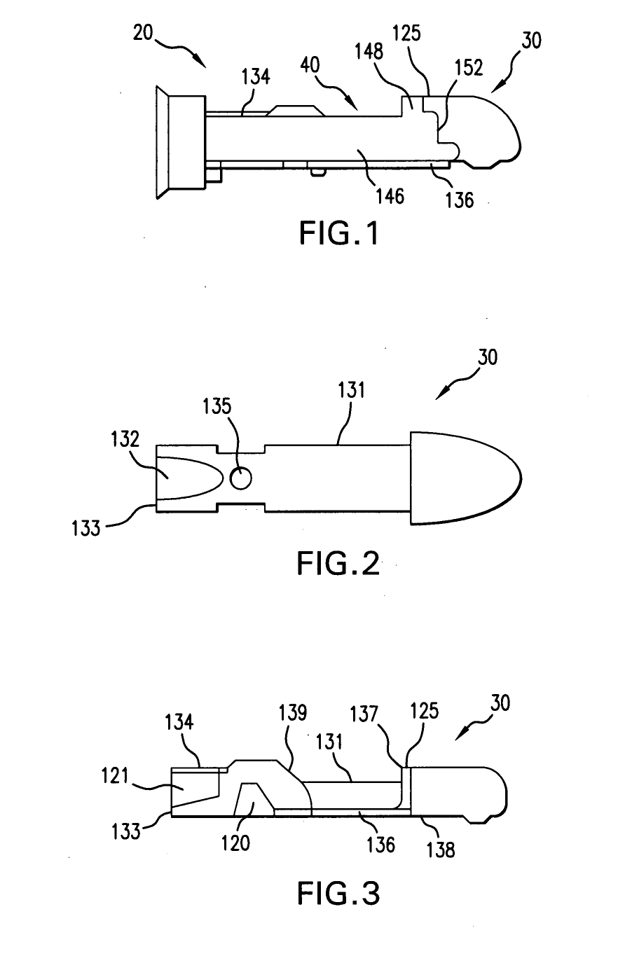

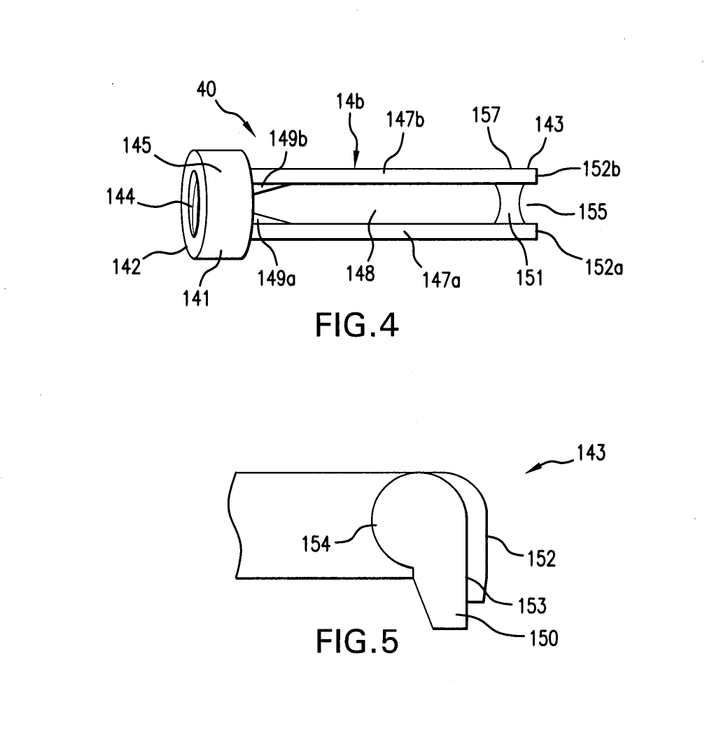

Referring to FIG. 1 in an embodiment of distal anchor 20, first member 30 is pivotally coupled to second member 40 via slidable engagement of first and second members 30 and 40. Specifically, referring to FIGS. 3 and 4, in this embodiment, first member 30 has a body 131 comprising a fitting portion 125 at a distal end 138 thereof. Second member 40 comprises a frame 146 defining a channel 148 sized to receive body 131 and a locking portion 143 at a distal end of second member 40. Locking portion 143 is configured to releasably engage fitting portion 125 such that first member 30 is in a radially un-extended position when fitting portion 125 is engaged by locking portion 143 and first member 30 can be moved to a radially extended position when fitting portion 125 is disengaged from locking portion 143. The fitting portion and the locking portion can comprise any type of cooperative structures that allow for the locking portion to releasably engage the fitting portion.

Referring to FIG. 2, body 131 of first member 30 can have a sloped, and preferably beveled, depression 132 at its proximal end 133 and a bore 135 extending from a top surface to a bottom surface of body 131 adjacent to depression 132. Referring to FIG. 3, first member 30 can further include at least one upper shoulder 134 disposed on a top surface of body 131 and at least one lower shoulder 136 disposed on a bottom surface of body 131. Preferably, the at least one upper shoulder 134 and the at least one lower shoulder 136 comprise an upper set of shoulders 134 and a lower set of shoulders 136, respectively. Lower shoulder 136 is adjacent to fitting portion 125 of body 131 near distal end 138 of body 131. In an embodiment, fitting portion 125 comprises a wall 137, which is adjacent to or abuts lower shoulder 136. First member 30 can further include at least one, and preferably two opposing sloping side portions 139 that slope from the top surface to the bottom surface of body 131 or lower shoulder 136. Referring still to FIG. 3, first member 30 can include at least one, and preferably two opposing tracks 120 on opposing exterior side surfaces thereof which run generally parallel to, and are preferably aligned with, bore 135 from the top surface to the bottom surface of body 131. First member 30 can also include at least one, and preferably two opposing tracks 121 on opposing exterior side surfaces near proximal end 133 of body 131 that run generally orthogonal to bore 135.

Referring to FIG. 4, in this embodiment of distal anchor 20, second member 40 comprises a head portion 141 at proximal end 142, a locking portion 143 at distal end 157, and a frame 146 extending between head portion 141 and locking portion 143.

Head portion defines an internally threaded bore 144 to receive a threaded portion of an insertion tool. Outer surface 145 of head portion 141 is sized to receive a spacer, which is used to maintain space between adjacent spinous processes, as described in more detail below.

Frame 146 of second member 40 includes opposing walls 147a and 147b that mutually define and border channel 148, which is in fluid communication with threaded bore 144. At least one, and preferably two opposing ribs 149a and 149b that are configured to slidably engage track 121 of first member 30 can be interiorly positioned on respective walls 147a and 147b, abut or be adjacent to head portion 141, and extend into channel 148.

Referring to FIGS. 4 and 5, in an embodiment, locking portion 143 of second member 40 includes a support arch 150 that defines a recess 151 and has a distal face 153. Support arch 150 can also include at least one, and preferably two opposing curved surfaces 154 that slope away from distal end 157 of second member 40 for slidable engagement with opposing sloping side portions 139 of first member 30. Locking portion 143 can also include one or more support projections 152a and 152b that extend from the distal end of frame 146. Support projections 152 and distal face 153 of support arch 150 mutually define groove 155 configured to receive wall 137 of first member 30 and releaseably lock first member 30 in a radially-unextended position. Wall 137 and groove 155 can interact, for example, by frictional engagement or by interference fitting where wall 137 is dimensioned slightly larger than groove 155 such that upon application of slight force, wall 137 will deform slightly upon placement in groove 155 for secure connection between first and second members 30 and 40.

In this embodiment, first member 30 and second member 40 interact with each other such that in a non-deployed configuration, first member 30 is releasably locked in a radially un-extended position and is generally aligned with the longitudinal axis of threaded bore 144 of second member 40. In a deployed configuration, first member 30 can slide to an extended position generally perpendicular to the longitudinal axis of bore 144. Specifically, referring to FIG. 1, in a non-deployed configuration, first member 30 is retained between walls 147 of frame 146. Shoulders 134 and 136 of first member 30 engage the top and bottom surfaces of walls 147 of second member 40 to support and contain first member 30 within channel 148 of second member 40. Fitting portion 125 of first member 30 is locked into locking portion 143 of second member 40. Specifically, in an embodiment, wall 137 of first member 30 is received by groove 155 of second member 40 and mates with supporting projection 152 and distal face 153 of support arch 150 to lock first member 30 in a radially un-extended position. In embodiments with ribs 149 on second member 40 and tracks 121 on first member 30, tracks 121 receive ribs 149 to prevent rotation of first member 20 in a non-deployed configuration.

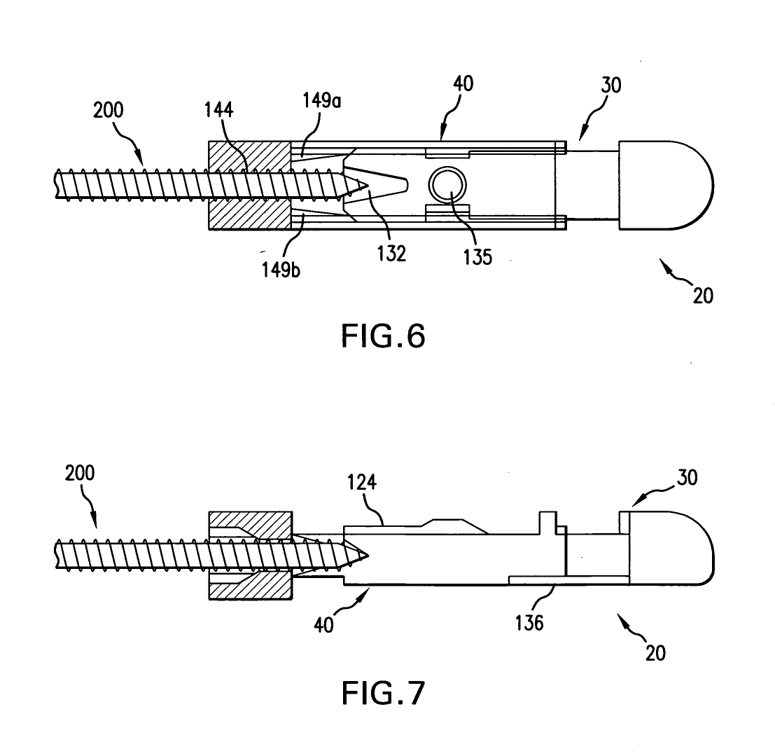

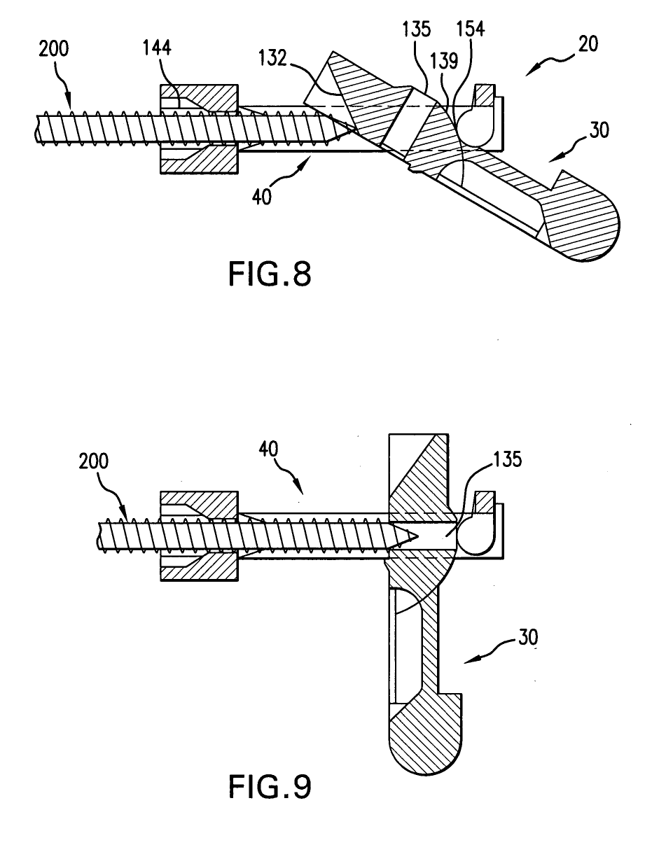

Referring to FIG. 6, to deploy first member 30, an insertion tool 200 having at least a threaded distal portion is inserted in threaded bore 144 of second member 40 for threadable engagement with bore 144 and advanced through bore 144. As the leading end of insertion tool 200 exits bore 144, it contacts depression 132 on body 131 of first member 30. As insertion tool 200 engages the sloped surface of depression 132, first member 30 disengages from the locked position, moving along a line parallel to the longitudinal axis of channel 148 along tracks 121 of first member 30. Referring to FIG. 7 and 8, as insertion tool 200 continues to advance through channel 148, the leading end of the insertion tool 200 pushes first member 30, which is guided by shoulders 134 and 136, until sloped side portion 139 of body 131 contacts curved surface 154 of support arch 150 of second member 40. Advancement of first member 30 along a line parallel to the longitudinal axis of channel 148 is prevented and as the leading end of insertion tool 200 advances along sloped depression 132, it urges first member 30 to pivot, following the arc of sloped side portion 139 in slidable communication with curved surface 154. Referring to FIG. 9, as a result, this embodiment of first member 30 is guided to a position generally orthogonal to the longitudinal axis of channel 148. Once first member 30 is rotated to this position, the edges of shoulder 134 and 136 provide support for first member 30. As insertion tool 200 continues advancing through channel 148, the leading end of insertion tool 200 advances through bore 135 of first member 30, securing first member 30 in a radially extended position.

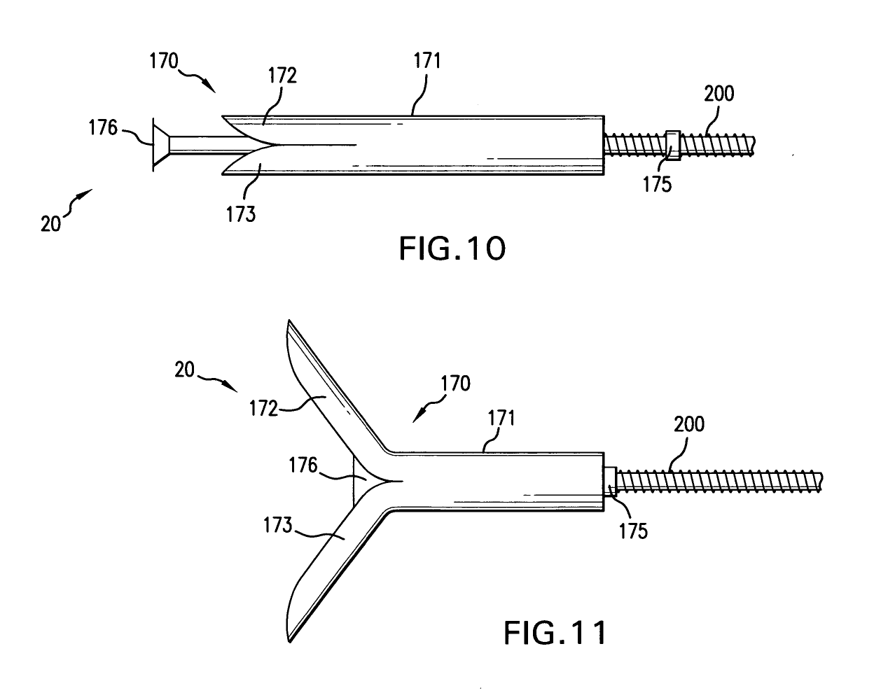

Although the above-described embodiments of a distal anchor involve a first member pivotally coupled to a second member by slideable engagement, the first member can be pivotally coupled to the second member by any other type of pivot mechanism such as a pivot pin or other types of pivots that effect rotational and/or sliding motions. The present invention also contemplates other types of distal anchors with other types of locking sections, including, for example, a first member that is hingedly coupled to a second member. The locking section can also comprise a deformable member that is moveable from a radially un-extended position to a radially-extended position. For example, referring to FIGS. 10 and 11, in an embodiment, distal anchor 20 comprises an anchor assembly 170 including a tubular member 171 axially splitting into at least two distal limbs 172 and 173 and defining a bore (not shown) extending therethrough. Anchor assembly 170 also includes wedge 176. In use, an insertion tool 200 is passed through the bore of tubular member 171 and wedge 176 is fixed on insertion tool 200 distal to limbs 172 and 173. Nut 175 is threaded onto insertion tool 200 and rotated to move distally along insertion tool 200 to contact and urge tubular member 171 against wedge 176, splaying limbs 172 and 172, as shown in FIG. 11.

In any of the above-described, the distal anchor can comprise any biologically compatible material, such as titanium, stainless steel, or a polymeric material such as polytetrafluoroethylene (PTFE) or polyetheretherketone (PEEK). In embodiments where the distal anchor is deformable, the distal anchor is fabricated from a deformable material such as, for example, stainless steel, nitinol, PTFE, or PEEK.

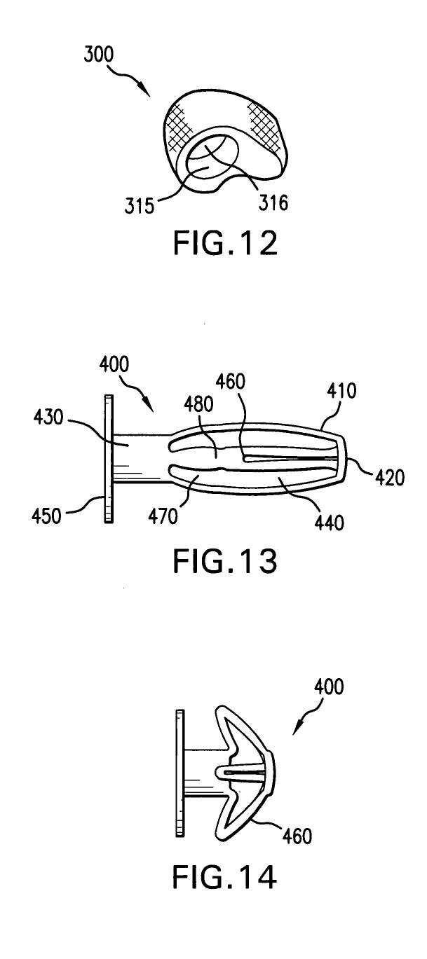

In an embodiment, an interspinous distraction kit of the present invention further comprises a spacer configured to maintain a space between adjacent spinous processes. Specifically, the spacer is sized and shaped to fit between and engage the superior surface of the inferior or caudal spinous process and the inferior surface of the superior or rostral spinous process. Referring to FIG. 12, spacer 300 has an inner surface 315 defining an opening 316 therethrough configured to receive the distal anchor. Specifically, in reference to FIG. 4, inner surface 315 defining opening 316 is configured to receive outer surface 145 of head portion 141 of the second member of the distal anchor. In a preferred embodiment, spacer 300 has a tear-drop cross-sectional configuration, as shown in FIG. 12, to fit against the contour of the spinous processes. Of course, the configuration of the spacer illustrated in FIG. 12 is only exemplary and the spacer can have other shapes such elliptical, egg-shaped, round-shaped, or saddle-shaped. The spacer can be made of any biologically acceptable material such as stainless steel, titanium, a polymeric material, or a super-elastic material or silicone, for example. The spacer can be made from a deformable material so that it can be urged into place, conform to the shape of the upper and lower spinous processes, and distribute the load forces between the spacer and the spinous processes. Further, a deformable spacer allows the spacer to mold to an irregular spinous process shape in order to position itself relative to the spinous process. By way of example only, the spacer can have a height of between 6-20 millimeters. Such heights refer to the maximum rostral to caudal height by which the spacer distracts and maintains apart the spinous processes.

In other embodiments of the present invention, an interspinous distraction kit further comprises a proximal anchor configured to lock against the proximal side of spinous processes to secure a spacer in the interspinous space. In preferred embodiment, the proximal anchor is moveable from a radially un-extended position in a non-deployed configuration to a radially extended position in a deployed configuration. Referring to FIG. 13, in an embodiment, the proximal anchor 400 comprises a tubular body 410 having a distal threaded opening 420, a proximal opening 430 and a lumen 440 extending therethrough. Preferably, proximal anchor 400 has a flange 450 at a proximal end thereof. Between proximal opening 430 and distal opening 420, tubular body 410 comprises a plurality of axially extending deformable strips 460, which define a plurality of axially extending slits 470 in fluid communication with lumen 440. Preferably, strips 460 are uniformly circumferentially spaced about body 410. When proximal anchor 400 is compressed axially, strips 460 will thereby be bended and yield laterally as shown in FIG. 14. As shown in FIG. 14, strips 460 can be formed with weakened notches 480 at predetermined positions that will cause strips 460 to have a tendency to bend or fold at those positions during compression.

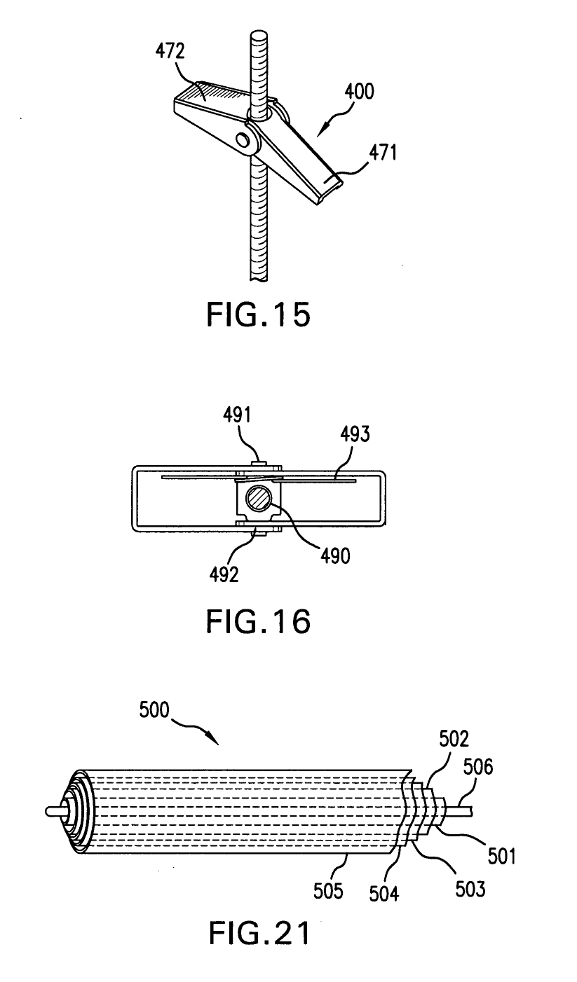

Referring to FIGS. 15 and 16, in another embodiment, proximal anchor 400 comprises opposing toggle wings 471 and 472 hinged coupled to one another. Toggle wings 471 and 472 can be hingedly coupled to one another, for example, by a trunnion nut 490 having a pair of coaxial opposed trunnions 491 and 492. Surrounding a trunnion of trunnion nut 490 is a coiled spring 493, the opposite ends of which act as opposing springs to open toggle wings 470 and 480.

Other proximal anchors are also envisioned by the present invention such as wings, flanges, collars, expansion anchors such as single and double expansion anchors and other locking mechanisms by which the proximal anchor can lock against the proximal side of spinous processes to secure a spacer in the interspinous space. The proximal anchor can comprise any biologically compatible material, such as titanium, stainless steel, or a polymeric material. Furthermore, embodiments of the proximal anchor of the present invention could be used as the distal anchor and embodiments of the distal anchor could be used as the proximal anchor. For example, the proximal anchor comprising opposing toggle wings described above could be used as a distal anchor.

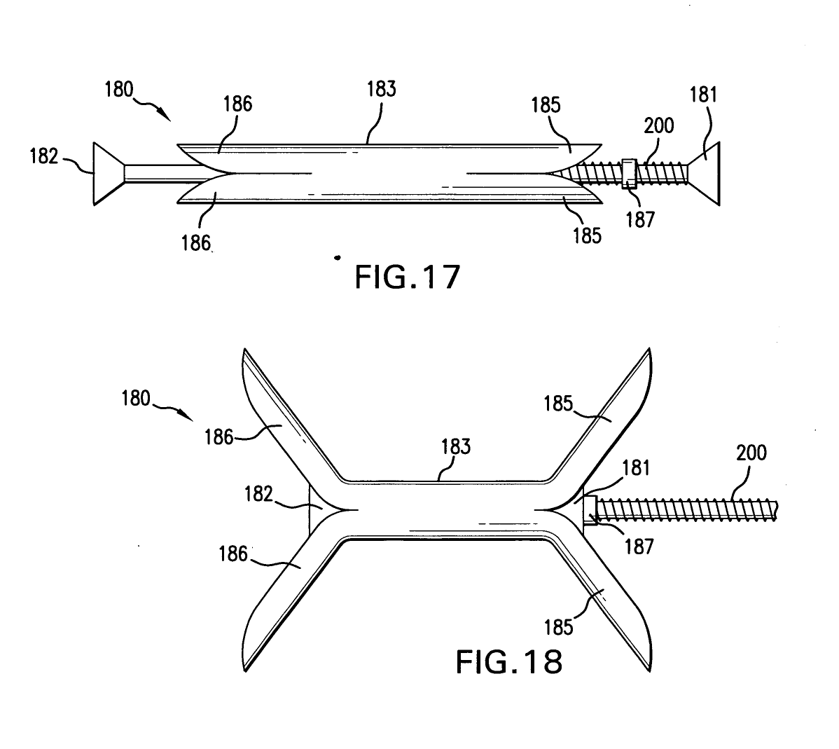

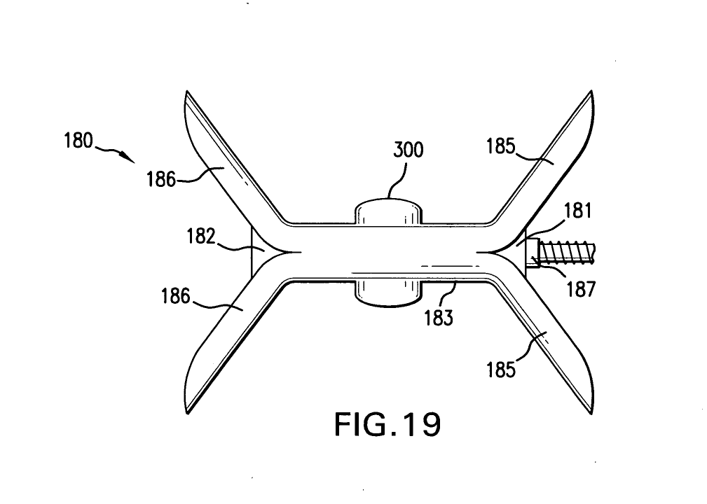

Although the above-described embodiments of a distal anchor, a proximal anchor, and a spacer have been described as separate pieces, the present invention contemplates an interspinous distraction assembly comprising a single device that serves as both a distal anchor and a proximal anchor and/or a single device that serves as a distal anchor, a spacer and a proximal anchor. For example, referring to FIGS. 17 and 18, in an embodiment, an interspinous distraction assembly 180 comprises a proximal wedge 181 defining an opening therethrough and a distal wedge 182. Interspinous distraction assembly 180 further comprises a tubular member 183 axially splitting into at least a pair of limbs 185, 186 at each end thereof and defining a bore (not shown) extending therethrough. In use, an insertion tool 200 is passed through the bore of tubular member 183, proximal wedge 181 is placed about insertion tool 200 proximal of limbs 185 and distal wedge 182 is fixed onto insertion tool 200 distal of limbs 186. Nut 187 is threaded onto insertion tool 200 and rotated to move distally along insertion tool 200 to contact and urge first wedge 181 against tubular member 183, splaying limbs 185 as shown in FIG. 18. Nut 187 is further tightened to urge tubular member 183 against second wedge 182 splaying limbs 186, as shown in FIG. 18. In such an embodiment, limbs 185 serve as a proximal anchor to abut against the proximal surface of the spinous processes and limbs 186 serve as a distal anchor to abut against the distal surfaces of the spinous processes. A separate spacer can be placed about tubular member 183 between limbs 185 and 186 or the spacer could be integrally formed with tubular member 183, as illustrated in FIG. 19. As seen in this figure, the central portion 300 of tubular member 183 itself serves as a spacer.

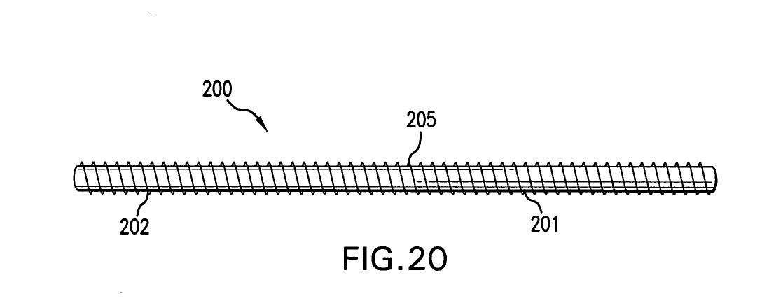

In other embodiments of the present invention, an interspinous distraction kit further comprises an insertion tool to insert an interspinous distraction assembly. Referring to FIG. 20, in an embodiment, an insertion tool 200 comprises a shaft 205 having an at least partially threaded portion 201 and a frangible portion 202. Frangible portion 202 allows insertion tool 200 to be cut or broken off after insertion of an interspinous assembly. The at least partially threaded portion 201 allows insertion tool to cooperate with the threaded portions of the distal and proximal anchors as described above. Of course, as one skilled in the art will appreciate, the insertion tool could employ other cooperative mechanisms, such as ratchet teeth, for example, to cooperate with portions of the distal and/or proximal anchor. Shaft 205 has a diameter such that it can pass through the lumen of a proximal anchor according to any of the embodiments of the present invention and the opening of a spacer according to any of the embodiments of the present invention. Further, for the applicable embodiments described above, shaft 205 has a diameter such that it can pass through threaded bore 144 of second member 40 of distal anchor 20 and threadably engage bore 135 of first member 30 as described above.

In other embodiments of the present invention, an interspinous distraction kit further comprises a dilator, such as a serial dilator to gradually enlarge the interspinous space prior to insertion of the spacer. Referring to FIG. 21, in an embodiment, a serial dilator 500 comprises a first dilator 501 having an inner diameter that is slightly larger than the outer diameter of a guide needle 506, which is used to pierce the interspinous ligament. Serial dilator 500 further comprises a second dilator 502 having an inner diameter that is slightly larger than the outer diameter of first dilator 501. Serial dilator can also have additional dilators, such as a third dilator 503, a fourth dilator 504, and a fifth dilator 505, each having successively larger inner diameters. The number of dilators may, of course, vary depending on the desired height of the interspinous space. In a preferred embodiment, the dilators of serial dilator 500 have a cross-sectional shape, at least at their distal end that correspond to the cross-sectional shape of the spacer. For example, if the spacer has a tear-drop cross-sectional configuration, then the serial dilator can have a tear-drop cross-sectional configuration. In use, as each dilator enters the interspinous space, it gradually and incrementally enlarges the height of the interspinous space until the interspinous space is at the desired height. Once the dilators are in place and the interspinous space is at the desired height, the guide needle and all the dilators, except the outermost dilator are removed. An intervertebral assembly including a distal anchor, a spacer and a proximal anchor is passed through the outermost dilator by an insertion tool.

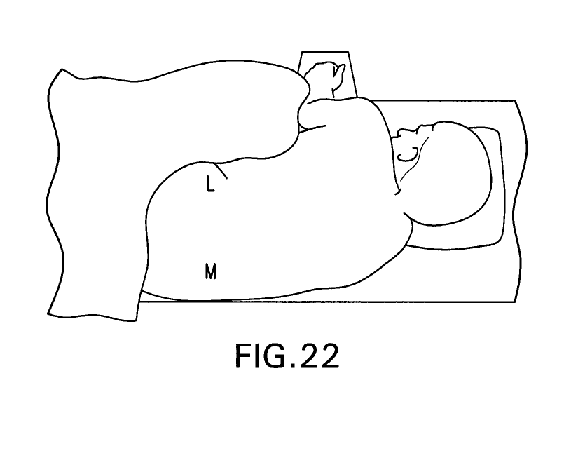

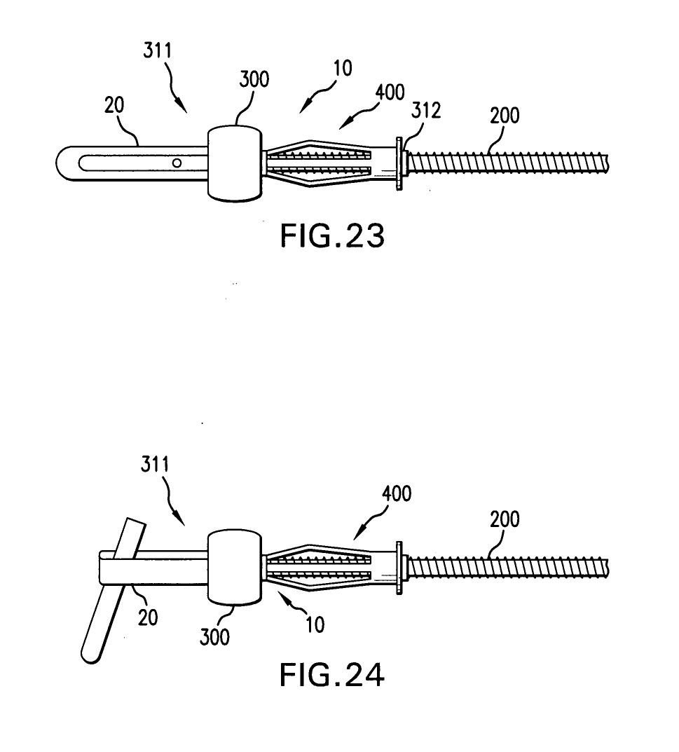

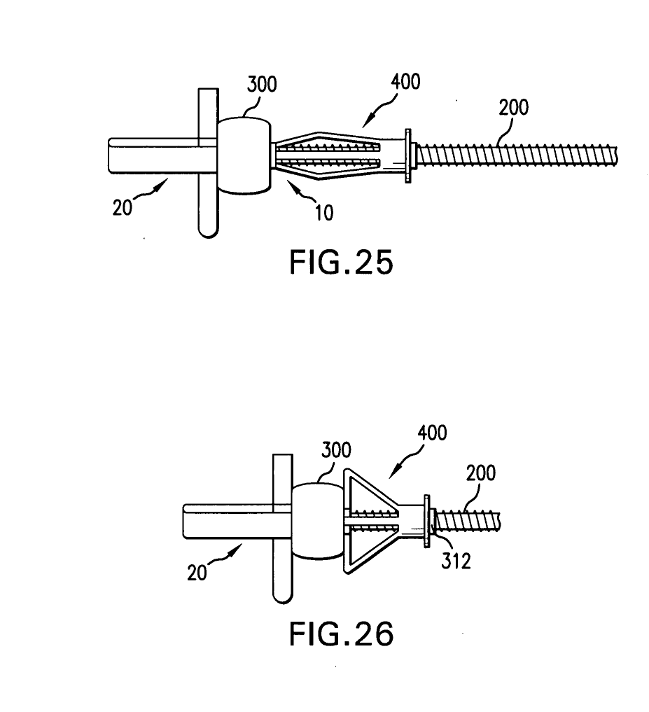

A non-limiting example of using an embodiment of an interspinous assembly to maintain a space between adjacent spinous processes will now be described with reference to FIGS. 22-26. Referring to FIG. 22, the patient is positioned in flexion, i.e. in the prone or lateral decubitus position, and a small incision is made in the lateral portion (L) of the patient's body, such as the patient's flank. A Kishner wire is advanced from lateral to medial through the interspinous ligament as anteriorly as possible, such as behind the facet joints. After the K wire is across the midline (M) and in the interspinous space, serial dilation is then carried out to dilate the interspinous space up to its maximum tension, such as, for example, 8-14 millimeters. Referring to FIG. 23, an interspinous assembly 10 according to an embodiment of the present invention is then assembled. Specifically, an appropriately-sized spacer 300 is affixed to a distal anchor 20 by any fixation method such that spacer is securely attached to distal anchor 20. Then the distal anchor/spacer assembly 311 and a proximal anchor 400 are sequentially placed about an insertion tool, such as a threaded rod 200. A locking nut 312 is also loosely threaded onto the threaded rod and positioned proximal to interspinous assembly 10. Interspinous assembly 10 is advanced through the appropriate dilation tube (i.e. one that is slightly larger then the selected spacer) and advanced across the midline of the patient's body. Referring to FIGS. 24 and 25, once spacer 300 is positioned in the interspinous space, threaded rod 200 is turned to deploy distal anchor 20 to lock interspinous assembly 10 against the contralateral surface of the spinous processes. Referring to FIG. 26, locking nut 312 is then tightened to deploy proximal anchor 400 locking interspinous assembly 10 against the ipsilateral surface of the spinous process. Spacer 300 is now securely captured within the interspinous space. Threaded rod 200 is either cut or broken off at the interface with locking nut 312. A single stitch or steri-strip is then used to close the incision in the patient's flank. Such an interspinous assembly and method of using the same allows a surgeon to implant a spacer in the interspinous space without having to make a bilateral incision at the patient's midline to access the contralateral side of the spinous processes to lock a distal anchor in place. The above-described method has been described with respect to a specific embodiment of an interspinous assembly of the present invention but the above-described method can be used with other types of interspinous assemblies, which allow the surgeon to access the spinous processes unilaterally.

The interspinous distraction kits and method of the present invention can be used, for example, for any condition where the patient complains of leg pain or back pain which is accentuated in lumbar spinal extension, such as standing or walking, and relieved by sitting or lumbar spinal flexion. Such conditions include, for example, spinal stenosis, lumbar facet joint syndrome, lumbar facet synovial cyst formation, painful internal disc disruption with posterior annular tear, and segmental stability.

The foregoing description and example have been set forth merely to illustrate the invention and are not intended as being limiting. Each of the disclosed aspects and embodiments of the present invention may be considered individually or in combination with other aspects, embodiments, and variations of the invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. An interspinous distraction kit comprising:
a distal anchor comprising a first member pivotally coupled to a second member, the first member moveable from a radially un-extended position in a non-deployed configuration to a radially extended position in a deployed configuration; and
a spacer having an inner surface defining an opening therethrough, the spacer configured to maintain a space between adjacent spinous processes.

2. The interspinous distraction kit of claim 1, wherein the first member is pivotally coupled to the second member by slidable engagement with the second member.

3. The interspinous distraction kit of claim 2, wherein the distal anchor comprises:
a first member having a body comprising a fitting portion at a distal end thereof; and
a second member comprising a head portion at a proximal end, a locking portion at a distal end, and a frame defining a channel disposed between the head portion and the locking portion, the channel receiving the body of the first member, wherein the locking portion of the second member is configured to releasably engage the fitting portion of the first member.

4. The interspinous distraction kit of claim 3, wherein the fitting portion comprises a wall and the locking member comprises a groove.

5. The interspinous distraction kit of claim 4, wherein the groove is mutually defined by a distal face of the frame of the second member and a support projection extending from the distal end of the frame.

6. The interspinous distraction kit of claim 3, wherein the first member comprises an upper and lower set of shoulders supporting the body of the first member within the channel of the second member.

7. The interspinous distraction kit of claim 3, wherein the proximal end of the body of the first member comprises a sloped depression.

8. The interspinous distraction kit of claim 7, wherein the body of the first member defines a bore extending from a top surface to a bottom surface thereof.

9. The interspinous distraction kit of claim 1, further comprising a proximal anchor.

10. The interspinous distraction kit of claim 9, wherein the proximal anchor is moveable from a radially un-extended position in a non-deployed configuration to a radiallly extended position in a deployed configuration.

11. The interspinous distraction kit of claim 10, wherein the proximal anchor has a body comprising a plurality of axially extending deformable strips defining a plurality of axially extending slits.

12. The interspinous distraction kit of claim 9, wherein the proximal anchor comprises opposing toggle wings hingedly coupled to one another.

13. The interspinous distraction kit of claim 12, wherein the opposing toggle wings are hingedly coupled to one another by a trunnion nut having a pair of coaxial opposed trunnions, the proximal anchor further comprising a coiled spring surrounding one of the trunnions.

14. The interspinous distraction kit of claim 1, wherein the spacer is tear-drop shaped.

15. The interspinous distraction kit of claim 1, further comprising an insertion tool having a threaded portion and a frangible portion.

16. The interspinous distraction kit of claim 1, further comprising a serial dilator comprising a plurality of dilators having sequentially larger inner diameters.

17. An interspinous distraction kit comprising:
a distal anchor comprising a first locking section moveable from a radially un-extended position in a non-deployed configuration to a radially extended position in a deployed configuration; and
a spacer having an inner surface defining an opening therethrough, the spacer configured to be maintain a space between adjacent spinous processes.

18. The interspinous system of claim 17, further comprising a proximal anchor comprising a second locking section moveable from a radially un-extended position in a non-deployed configuration to a radially extended position in a deployed configuration.

19. An interspinous distraction kit comprising:
a distal anchor comprises a tubular member axially splitting into at least two limbs;
a wedge; and
a spacer configured to maintain a space between adjacent spinous processes.

20. The interspinous distraction kit of claim 19, further comprising a proximal anchor.

21. The interspinous distraction kit of claim 20, wherein the proximal anchor is moveable from a radially un-extended position in a non-deployed configuration to a radiallly extended position in a deployed configuration.

22. An interspinous distraction assembly comprising:
a tubular member axially splitting into at least a pair of proximal limbs and at least a pair of distal limbs, the tubular member defining a bore extending therethrough;
a proximal wedge defining an opening therethrough;
a distal wedge; and
a spacer positioned on the tubular member between the proximal limbs and the distal limbs.

23. The interspinous distraction assembly of claim 22, wherein the spacer is integrally formed with the tubular member.

24. A method for maintaining a space between adjacent spinous processes comprising:
incising a lateral portion of the patient's body;
advancing an interspinous assembly towards the midline of the patient's body, the interspinous assembly comprising a spacer, a distal anchor, and a proximal anchor;
positioning the spacer in the interspinous space between adjacent spinous processes;
deploying the distal anchor to secure the distal anchor against the distal surface of the adjacent spinous processes; and
deploying the proximal anchor after deployment of the distal anchor to secure the proximal anchor against the proximal surface of the adjacent spinous processes.

25. The method of claim 24, wherein the lateral portion of the patient's body is the flank.
